(19) United States
(12) Patent Application Publication
(10) Pub. No.: US 2020/0061219 A1
(43) Pub. Date: Feb. 27, 2020

(54) MAGNETIC ORGANIC CORE-INORGANIC SHELL MATERIAL, PROCESS FOR PRODUCING SAME AND USES THEREOF FOR THE MAGNETICALLY STIMULATED DELIVERY OF SUBSTANCES OF INTEREST

(71) Applicants: CENTRE NATIONAL DE LA RECHERCHE SCIENTIFIQUE, PARIS (FR); INSTITUT POLYTECHNIQUE DE BORDEAUX, TALENCE (FR); UNIVERSITE DE BORDEAUX, BORDEAUX (FR)

(72) Inventors: Véronique SCHMITT, TALENCE (FR); Rénal BACKOV, BORDEAUX-CAUDERAN (FR); Marion BAILLOT, BORDEAUX (FR); Olivier SANDRE, PESSAC (FR)

(21) Appl. No.: 16/466,222

(22) PCT Filed: Dec. 4, 2017
(86) PCT No.: PCT/FR2017/053378

§ 371 Date: Jun. 3, 2019

(30) Foreign Application Priority Data
Dec. 5, 2016 (FR) 1661952

(51) Int. Cl. A61K 49/18; A61K 41/00

(52) U.S. Cl. CPC A61K 49/1839; A61K 41/00

(57) ABSTRACT

The present invention relates to a submicrometric material consisting of a silica shell encasing a core of superparamagnetic wax, to the process for producing same and to the uses thereof, in particular for the magnetically stimulated delivery of substances of interest.

The present invention relates to a submicrometric material consisting of a silica shell encasing a core of superparamagnetic wax, to the process for producing same and to the uses thereof, in particular for the magnetically stimulated delivery of substances of interest.

It is known practice to encapsulate molecules of interest such as drugs, dyes, pigments, reagents, fragrances, pesticides, etc., in order to protect them against outside attacks, in particular oxidation, in order to convey them to a site of administration where they will be able to be delivered or else in order to store them before use under conditions where they will be released from their capsule under the influence of an internal or external stimulus. One of the first applications of microencapsulation was the development of a carbonless copy paper sold at the end of the 1960s, in which microcapsules enclosing an ink were present on the back of a sheet of paper so as to release the ink by rupture of the capsules under the pressure exerted by the tip of a pen when writing. These days, encapsulation is expanding in various industrial sectors, such as the pharmaceutical, cosmetic, food, textile and agricultural industries. The capsules and microcapsules are becoming increasingly sophisticated, in particular in the pharmaceutical field where they make it possible to carry out controlled and/or targeted delivery of one or more active ingredients.

Various types and morphologies of capsules have already been proposed, such as, for example, protein capsules, peptide capsules, cyclodextrins, heat-sensitive liposomes, polymerosomes, colloidosomes, silica-shell microcapsules, nanocapsules comprising a silica core and a shell of heat-sensitive polymer such as poly(N-isopropylacrylamide) (PNIPAM), or conversely a core of heat-sensitive polymer such as Pluronic® F68/poly(vinyl alcohol) and a silica shell, heat-sensitive hydrogel microspheres, PNIPAM-polylactide microspheres, etc. Numerous methods for preparing these various capsule types and morphologies have also been developed over the past few years, such as, for example and non-exhaustively, the precipitation of polymers by phase separation, layer-by-layer electrolyte deposition, polymerization by interfacial condensation, etc. Depending on the type and morphology of the capsules developed, the release of the molecules of interest may be slow and gradual (that is to say sustained over time) or provoked (that is to say triggered by an action). In particular, the release of the molecules of interest may be triggered under the effect of one or more internal and/or external stimuli such as, for example, a change in pH, a redox process, an enzymatic catalysis, ultrasound, use of specific agents such as foaming agents, a change in temperature, a light irradiation, near-infrared radiation, a modification of osmotic pressure, disruption of the coating by swelling of the core, an electric field or a magnetic field.

In particular, patent application FR-A-2 948 581 describes a micrometric material (12.5-50 μm) consisting of a silica shell encasing a wax core containing one or more substances of interest, these materials being prepared by mineralization of a Pickering emulsion, that is to say an emulsion of oil-in-water type in which the dispersion of the oil droplets in water is stabilized by colloidal nanoparticles adsorbed at the water/oil interface. By using a crystallizable oil, that is to say an oil of which the melting point (M_{P}) is quite low (for example 37° C.), it is possible to prepare a material in which the encapsulated phase (i.e. the core) is solid at ambient temperature but becomes liquid when said material is heated by means of a hotplate to a temperature of about 50-60° C., thus causing breaking of the capsule by melting and thermal expansion of the encapsulated phase and concomitant and rapid release of the substance(s) of interest contained in the encapsulated phase.

However, this material is micrometric in size, which does not make it possible to use it in certain fields of application, such as the field of nanodrugs (e.g. intravenous injection of nanodrugs) or of nanocosmetics (e.g. vesicles or capsules encasing a fragrance, a vitamin, an antioxidant). Moreover, the use of a macroscopic external heat source (e.g. hotplate) is not suitable for the release of the molecules of interest in in vivo applications. Furthermore, the temperatures and the heating rates used to enable the breaking of the silica shell are too high for in vivo applications and/or certain heat-sensitive environments. Furthermore, they consume energy. In particular, the silica core is an excellent thermal insulator; it is therefore difficult to get through the adiabatic chamber constituted by said shell, in particular by external heating. Finally, the control of the release of the molecules of interest is not optimized (e.g. release too rapid).

There is therefore at the current time no submicrometric system which allows a gradual release of molecules of interest under the effect of an internal or external stimulus under mild conditions, while at the same time guaranteeing better control of the release, in particular for in vivo applications.

The objective of the present invention is therefore to provide a submicrometric capsule which makes it possible to encapsulate one or more molecules of interest that can be released gradually under mild conditions, while at the same time guaranteeing better control of the release, in particular for in vivo applications.

The objective of the present invention is also to provide a process for preparing submicrometric capsules that is easy to carry out and economical, said process making it possible to encapsulate one or more molecules of interest that can be released gradually under mild conditions, while at the same time guaranteeing better control of the release, in particular for in vivo applications.

The first subject of the present invention is a material in the form of solid particles containing a fatty phase that is solid at the temperature of storage of said material and a continuous shell comprising at least one silicon oxide and enclosing said fatty phase, said fatty phase comprising a crystallizable oil having a melting point (M_{P}) less than approximately 100° C. and at least one substance of interest, said material being characterized in that it is submicrometric and in that the fatty phase also comprises superparamagnetic nanoparticles surface-functionalized with at least one fatty acid.

According to the present invention, the expression “temperature for storing said material” is intended to mean the temperature at which the material in accordance with the present invention is stored before it is used. This temperature is always lower than the melting point of the crystallizable oil contained in the fatty phase. It is preferably between −25 and 25° C. approximately, and more preferably between 0 and 22° C. approximately.

When said material is subjected to an alternating magnetic field, the functionalized superparamagnetic nanoparticles contained in the fatty phase heat up locally, which leads locally to the heating of the fatty phase to a temperature above the melting point of the crystallizable oil (M_{P}). A thermal expansion of the fatty phase is then observed, leading to breaking of the silica shell and gradual release of the molten fatty phase (i.e. in the liquid state) comprising the substance(s) of interest. Moreover, the local heating of the functionalized superparamagnetic nanoparticles is sufficient to enable the melting of the fatty phase, thus bringing about the release of the substance(s) of interest.

In the context of this disclosure, the term “crystallizable oil” is intended to mean fats and mixtures of fats, of natural (animal or plant) or synthetic origin, the melting point of which is greater than approximately 15° C., the melting point of which preferably varies from 20 to 100° C. approximately, and in particular from 20 to 50° C. approximately. All the melting points mentioned in the description of the present application refer to melting points determined by differential scanning calorimetry (DSC) at atmospheric pressure.

The crystallizable oil forms a major part of the fatty phase and may even, in addition to the substance(s) of interest and the functionalized superparamagnetic nanoparticles, be the only constituent thereof. Generally, the crystallizable oil represents at least 50% by weight approximately, preferably from 50% to 99.8% by weight approximately, and more preferably from 75% to 98% by weight approximately, of the fatty phase.

The choice of the crystallizable oil naturally depends on the envisaged application for the material and thus on the temperature at which it is desired to observe the thermal expansion of the fatty phase and, consequently, the breaking of the silica shell. Among the crystallizable oils that can be used according to the invention, mention may in particular be made of paraffins, such as paraffins having a melting point between 42 and 44° C. or between 46 and 48° C. [RN-8002-74-2], in particular sold by the company Merck; triglycerides; fatty acids such as dodecanoic acid, also known as lauric acid, the melting point of which is 43.2° C.; rosins; waxes (long alkanes, i.e. comprising at least 12 carbon atoms), such as eicosane or octadecane; synthetic bitumens and hydrogenated plant oils and also mixtures thereof. These oils may be used alone or as mixtures.

Waxes are preferred.

The crystallizable oil preferably has a melting point (M_{P}) of between 30 and 60° C. approximately, and preferably of between 30 and 40° C. approximately.

The material in accordance with the present invention is submicrometric. In other words, it is in the form of a suspension of solid submicrometric particles dispersed in an aqueous phase or of a powder of solid submicrometric particles.

Moreover, each of the submicrometric particles of the material is a submicrometric capsule (since each of the submicrometric particles comprises a silica shell and a fatty phase enclosed in said silica shell).

The submicrometric particles of said material of the invention are preferably spherical or substantially spherical.

The diameter of the submicrometric particles (or of their smallest dimension in the case where they are not spherical) is less than 1 μm approximately, preferably ranges from 400 nm to 900 nm approximately, and even more preferentially from 700 to 850 nm approximately.

The submicrometric particles constituting said material are preferably monodisperse. They therefore preferably have a narrow particle size distribution of the particle diameters and in particular a polydispersity index with respect to size of at most approximately 0.1, and preferably of at most approximately 0.07. The polydispersity can be measured by scanning electron microscopy (SEM) which gives a dimensionless standard deviation, and/or by quasi-elastic light scattering (QELS) which gives a polydispersity index (PDI) which is equal to the square of the dimensionless standard deviation [J. Chem. Phys., 1972, 57, 11, 4814-4820]. The measurement of the size dispersion can be carried out by calculating the dimensionless standard deviation, which is the ratio of the standard deviation of the size distribution to the mean diameter, from a histogram of size obtained by measuring the individual diameters of a set of submicrometric particles (minimum 500) on one or more SEM images preferably with a magnification of 8000, or by measurement of dynamic light scattering which gives the mean hydrodynamic diameter and the PDI, from which it is possible to deduce the dimensionless standard deviation by taking its square root.

The superparamagnetic particles collectively have a zero magnetization at zero field and the magnetization induced by application of a magnetic field is virtually proportional to the field applied over the whole of the first part of the curve of their magnetization as a function of the field applied. They can therefore lead to the formation of a suspension of which the stability is not disrupted by the dipolar magnetic attraction between the magnetic moments of the nanometric particles, the suspension of the latter having no spontaneous magnetization in the absence of magnetic field. In other words, superparamagnetic nanoparticles have the double advantage of being able to undergo a strong attraction by a magnet or a magnetic field, and of not aggregating in the absence of magnetic field (i.e. the absence of spontaneous magnetization in zero field).

In the invention, the expression “nanoparticles” means that at least 50% of the distribution by number of said nanoparticles have a diameter of less than 100 nm.

The diameter of the superparamagnetic nanoparticles contained in the fatty phase preferably ranges from 10 to 20 nm approximately, and even more preferentially from 12 to 16 nm approximately.

The abovementioned diameter ranges, an optimal specific adsorption rate in a radio frequency magnetic field can be obtained, in particular for superparamagnetic iron oxides.

The superparamagnetic nanoparticles are preferably homogeneous with respect to size, and more preferably monodisperse.

The superparamagnetic nanoparticles which are homogeneous with respect to size or monodisperse have optimal heating properties by external application of a radio frequency oscillating magnetic field.

The size distribution of the superparamagnetic nanoparticles is generally measured by transmission electron microscopy (TEM) or by small-angle neutron scattering (SANS). The measurement of the size dispersion can be carried out by calculating the ratio of the standard deviation of the size distribution to the mean diameter from a histogram of size obtained by measuring the individual diameters of a set of nanoparticles (minimum 500) on one or more TEM images with a magnification of preferably 80 000 and/or by adjustment of the curve of the scattered intensity by SANS, by convolution of a spherical-particle aspect ratio with a diameter distribution law.

A size homogeneity can for example be obtained by virtue of a process for size-sorting superparamagnetic nanoparticles, based on phase separations, which process will be described below.

In the present invention, the expression “nanoparticles homogeneous with respect to size” means nanoparticles having a polydispersity index with respect to size of at most approximately 0.5, and preferably of at most approximately 0.4.

In the present invention, the expression “monodisperse nanoparticles” means nanoparticles having a polydispersity index with respect to size of at most approximately 0.1, and preferably of at most approximately 0.05.

By virtue of the monodispersity or the homogeneity with respect to size of the superparamagnetic nanoparticles, the magnetic response is homogeneous (i.e. magnetic properties homogeneous over the whole of a batch of particles).

The superparamagnetic nanoparticles can be chosen from nanoparticles of a magnetic iron oxide, nanoparticles of a mixed oxide of iron and of another transition metal, and nanoparticles of a ferric oxide of spinel structure that has vacancies, having the chemical formula γ-Fe₂O₃ (commonly known as maghemite).

The nanoparticles of a magnetic iron oxide may be iron ferrite nanoparticles, also referred to as magnetite nanoparticles, of formula Fe₃O₄.

The nanoparticles of a mixed oxide of iron and of another transition metal may be nanoparticles of ferrite of chemical formula MO.Fe₂O₃ in which M denotes a transition metal of spinel structure different from iron, or nanoparticles of chemical formula M₁₋ₓM′ₓO.Fe₂O₃ in which M and M′ denote transition metals different from iron and 0<x<1.

M (respectively M′) can be chosen from manganese (Mn), zinc (Zn) and nickel (Ni).

According to one particularly preferred embodiment of the invention, the superparamagnetic nanoparticles are maghemite nanoparticles (of formula γ-Fe₂O₃).

The superparamagnetic nanoparticles contained in the fatty phase of the material of the invention are functionalized with at least one fatty acid. This functionalization is a functionalization by chemisorption.

In the present invention, the expression “fatty acid” means an aliphatic-chain carboxylic acid, preferably comprising from 4 to 36 carbon atoms, and more preferably from 8 to 22 carbon atoms. The fatty acid may be saturated or unsaturated, that is to say comprising one or more carbon-carbon double bonds. It may be denoted Cn:m where n denotes the number of carbon atoms and m the number of double bonds.

The fatty acid may be chosen from arachidic acid (C20:0), stearic acid (C18:0), oleic acid (C18:1), palmitic acid (C16:0), myristic acid (C14:0), lauric acid (C12:0), capric acid (C10:0) and caprylic acid (C8:0).

In certain cases, the fatty acid, in particular lauric acid, may consequently serve both as crystallizable oil and as functionalization agent (i.e. lipophilic stabilizer) for the superparamagnetic nanoparticles.

The saturated fatty acids such as stearic acid are preferred, in particular by virtue of their better stability in the face of degradation by photo- or thermooxidation or by hydroperoxidation, and by virtue of their melting point which is higher than those of unsaturated fatty acids of the same chain length.

In particular, the choice of a fatty acid of which the aliphatic chain has a chain length close or identical to that of the crystallizable oil promotes the dispersion of the superparamagnetic nanoparticles in the crystallizable oil and therefore a better control of the breaking of the silica shell for the release of a substance of interest. Consequently, stearic acid (C18:0) can advantageously be used to functionalize the superparamagnetic nanoparticles when the crystallizable oil is a wax such as eicosane. Specifically, eicosane contains 20 carbon atoms, and its chain length is thus close to that of stearic acid, while at the same time having a melting point (around 37° C.) lower than that of stearic acid (around about 50-69° C.).

The functionalization makes it possible in particular to obtain lipophilic superparamagnetic nanoparticles, each of the nanoparticles being coated with fatty acid molecules, in particular in the form of a self-assembled monolayer.

The fatty acid generally represents from 10% to 30% by weight approximately, and preferably from 20% to 25% by weight approximately, relative to the total weight of the functionalized superparamagnetic nanoparticles.

The functionalized superparamagnetic nanoparticle content of the fatty phase is such that it enables it to be locally heated to a temperature greater than its melting point M_{P}. In other words, the concentration by weight of the functionalized superparamagnetic nanoparticles in the fatty phase is preferably sufficient to induce solid-liquid phase transition of the crystallizable oil during the application of a radio frequency alternating magnetic field to the submicrometric capsules, while at the same time guaranteeing good colloidal stability when the superparamagnetic nanoparticles are dispersed in the liquid fatty phase before the preparation of the submicrometric capsules (e.g. step 1) of the process as described below).

In one particular embodiment, the functionalized superparamagnetic nanoparticles represent from 0.2% to 3% by weight approximately, and preferentially from 1% to 2.5% by weight approximately, of the total weight of the fatty phase.

The diameter of the fatty phase that is solid at the storage temperature of said material preferably ranges from 400 nm to 950 nm approximately, and even more preferentially from 450 to 825 nm approximately.

The fatty phase of the material in accordance with the invention can contain any type of substances of interest, whether they are lipophilic or hydrophilic. Thus, when the substance(s) of interest are lipophilic, the fatty phase contains them in solubilized form and when the substance(s) of interest are hydrophilic, the fatty phase contains them in disperse form (dispersed directly in the crystallizable oil or in a water fraction dispersed within the fatty phase (double emulsion)). They may also be solid particles.

Among the substances of interest that can be incorporated into the fatty phase of the material in accordance with the present invention, mention may in particular be made of drugs (active ingredients), active ingredients that can be used in cosmetics, chemical reagents, dyes, pigments, inks such as electronic or magnetic inks for displays or for coding and authentification processes (nonforgeable ink), etc.

By way of examples of drugs, mention may be made of bactericides such as antiseptics and antibiotics, anti-inflammatories (ibuprofen, budesonide), analgesics, local laxitives, hormones, proteins, anti-cancer agents (tamoxifen, paclitaxel), etc.

By way of examples of cosmetic active ingredients, mention may be made of vitamins (e.g. retinol), sunscreens, antioxidants such as free-radical scavenger compounds, for instance the superoxide dismutase enzyme, fragrances, odour absorbers, deodorants, antiperspirants, dyes, pigments, emollients, moisturizing agents, etc.

By way of examples of chemical reagents, mention may be made of coloured reagents, coloured indicators such as pH indicators, catalysts, polymerization initiators, monomers, complexing agents, etc.

The substance(s) of interest generally represent from 0.001% to 35% by weight approximately, and preferentially from 0.01% to 25% by weight approximately, of the total weight of the fatty phase.

The fatty phase may also contain one or more additives conventionally used in emulsions and among which mention may in particular be made, by way of examples, of surfactants, protectors or agents for preserving the substance of interest, such as antioxidants, anti-UV agents, etc.

The silica shell preferably has a thickness and a density that are sufficient to have a mechanical strength that allows the encapsulation of the fatty phase, while at the same time being fine enough and having a density that is low enough to be able to break during the application of a magnetic field leading to the local heating of the fatty phase constituting the core of the material via the superparamagnetic nanoparticles.

The thickness of the silica shell generally ranges from 30 to 50 nm approximately, and preferentially from 36 to 46 nm approximately.

The density of the silica shell generally ranges from 1.0 to 2.5 g/cm³, and preferentially from 1.3 to 2.3 g/cm³.

In addition to the silicon oxide, the shell may also comprise one or more metal oxides of formula MeO₂, in which Me is a metal chosen from Zr, Ti, Th, Nb, Ta, V, W and Al. In this case, the shell is a mixed matrix of SiO₂-MeO₂ type in which the weight content of MeO₂ remains in the minority compared with the content of silicon oxide, the weight content of MeO₂ preferentially representing from 1% to 40% approximately, more particularly from 5% to 30%, relative to the total weight of the shell.

A second subject of the invention is a process for producing a material as defined in the first subject of the invention. This process is characterized in that it comprises at least the following steps:

1) preparing a fatty phase in the liquid state comprising a crystallizable oil in the liquid state having a melting point M_{P} of less than approximately 100° C., at least one substance of interest and superparamagnetic nanoparticles functionalized with at least one fatty acid;

2) bringing said fatty phase in the liquid state of step 1) into contact with an aqueous phase (AP) brought beforehand to a temperature TAP such that TAP is greater than M_{P}, said aqueous phase (AP) containing colloidal solid particles;

3) subjecting the liquid mixture resulting from step 2) to mechanical stirring so as to obtain an oil-in-water (O/W) emulsion formed of droplets of fatty phase in the liquid state dispersed in a continuous aqueous phase and in which the colloidal solid particles are present at the interface formed between the continuous aqueous phase and the dispersed droplets of fatty phase;

4) leaving said O/W emulsion to stand and then cooling it to a temperature T_{O/W} such that T_{O/W} is less than M_{P} so as to bring about the solidification of the fatty phase and to obtain an O/W emulsion formed of globules of fatty phase in the solid state, said globules being dispersed in the continuous aqueous phase;

5) forming a shell comprising at least one silicon oxide around each of said globules by addition, to the continuous aqueous phase of the O/W emulsion of step 4), and with mechanical stirring, of at least one precursor of silicon oxide, of a surfactant SA₁ and of a sufficient amount of at least one acid to bring the aqueous phase to a pH of less than or equal to 4 so as to obtain said material;

6) optionally, separating said material from the aqueous phase.

The crystallizable oil used in step 1) is as defined in the first subject of the invention.

The superparamagnetic nanoparticles functionalized with at least one fatty acid and the substance of interest are as defined in the first subject of the invention.

Step 1) can be carried out according to either one of the following two methods:
bringing a fatty phase comprising a solid crystallizable oil having a melting point M_{P} of less than approximately 100° C. to a temperature T_{CO} such that T_{CO} is greater than M_{P}, so as to obtain a fatty phase in the liquid state; and incorporating, into the fatty phase in the liquid state of the preceding step, at least one substance of interest and superparamagnetic nanoparticles functionalized with at least one fatty acid (first method), or
mixing a fatty phase in the solid state comprising a solid crystallizable oil having a melting point M_{P} of less than approximately 100° C. with superparamagnetic nanoparticles functionalized with at least one fatty acid and at least one substance of interest; and bringing the resulting mixture to a temperature T_{CO} greater than M_{P}, so as to obtain a fatty phase in the liquid state (second method).

The functionalized supermagnetic nanoparticles and the substance of interest can be introduced (e.g. first method) or mixed (e.g. second method) together or separately. The second case may have an advantage in the case of a fragile substance for which the residence time at the temperature T_{CO} greater than M_{P} must be minimized.

It will advantageously be preferred to introduce the substance of interest into the fatty phase in the liquid state (first method) separately, and in particular last.

The colloidal solid particles present in the aqueous phase (AP) during step 2) may be mineral or organic. They are preferably mineral particles. The colloidal solid particles are preferably mineral particles chosen from the group of metal oxides, hydroxides and sulfates, the oxides being particularly preferred. Among such oxides, mention may most particularly be made of silicon, titanium, zirconium or iron oxides, and also the salts thereof such as the silicates (for example clays). Any other type of particles that are not strictly mineral (e.g. carbon black, fullerene, graphene, graphene oxide, boronene, etc.) can also be envisaged for stabilizing the interface.

In order to be colloidal, the solid particles generally have a size of less than a few micrometres. Thus, the particles generally have an average size of between 5 and 5000 nm approximately, and preferably between 5 and 500 nm approximately.

According to one particularly preferred embodiment of the invention, the colloidal solid particles are chosen from silicon oxide nanoparticles. By way of example, mention may be made of the products sold under the trade name Aerosil® by the company Evonik Degussa, such as the colloidal solid particles of silica having a diameter of 7 nm, sold under the reference Aerosil® A380.

The silicon oxide nanoparticles generally have an average size of between 5 and 12 nm approximately.

The amount of colloidal solid particles generally ranges from 0.5% to 1.7% by weight approximately, and preferably from 1.0% to 1.4% by weight approximately, relative to the total weight of the aqueous phase (AP).

According to one preferred embodiment of the invention, the colloidal solid particles are surface-functionalized so as to make them more hydrophobic. This thus makes it possible to promote their adsorption at the surface of the droplets of the dispersed fatty phase during step 3) (i.e. at the interface formed between the continuous aqueous phase and the disperse droplets of the fatty phase).

The colloidal solid particles can thus be functionalized with compounds bonded to their surface by covalent bonds (chemisorption) or by adsorption of molecules of a surfactant SA₂ at their surface by electrostatic bonds (physisorption).

The functionalization by chemisorption can be carried out by prior treatment of the colloidal solid particles, in particular by chemical grafting of a compound comprising hydrophobic groups, such as a trihalosilane or a trialkoxysilane of formula R—Si—(OR′)₃, in which R is a linear or branched alkyl having from 1 to 12 carbon atoms, in particular having from 2 to 10 carbon atoms, most particularly an n-octyl group, optionally bearing an amino group, and R′, which may be identical or different from R, is a linear or branched alkyl group having from 1 to 12 carbon atoms, in particular having from 1 to 6 carbon atoms, and most particularly an ethyl group.

By way of example of colloidal solid particles functionalized by chemical grafting of a compound comprising hydrophobic groups (e.g. silane compound), mention may in particular be made of the silica nanoparticles 12 nm in diameter, treated with dichlorodimethylsilane, sold under the name Aerosil® R816 by the company Evonik Degussa and the silica nanoparticles 16 nm in diameter, treated with hexadecylsilane, sold under the name Aerosil® R972 by the company Evonik Degussa.

The functionalization by physisorption makes it possible to confer a certain hydrophobicity on the colloidal solid particles, the hydrophilic end of the SA₂ surfactant being adsorbed onto the surface of the particles. The SA₂ surfactants that can be used to functionalize the particles are preferably cationic or anionic surfactants.

Among these SA₂ surfactants, sodium alkyl sulfates, such as sodium dodecyl sulfate (SDS), aliphatic and/or aromatic sodium sulfonates, such as sodium dodecylbenzylsulfonate (SDBS), or alkyltrimethylammonium bromides, such as hexadecyltrimethylammonium bromide (CTAB), are in particular preferred.

The SA₂ surfactant is preferably chosen from surfactants having a charge opposite to that of the surface of the colloidal surface particles. This choice makes it possible to promote the adsorption of the SA₂ surfactant at the surface of the particles.

Functionalization of the colloidal solid particles by an SA₂ surfactant can also be carried out in situ, that is to say during their introduction into the aqueous phase (AP). In this case, the aqueous phase (AP) also contains said SA₂ surfactant in a concentration preferably lower than the critical micelle concentration (CMC) of said SA₂ surfactant, said surfactant then adsorbing at the surface of the colloidal solid particles when the latter are in the aqueous phase (AP). Preferably, the amount of SA₂ surfactant ranges from 1/200 to ⅔ approximately of the CMC.

According to one embodiment, the weight of SA₂ surfactant weight/weight of colloidal solid particles ratio by weight ranges from 0.015 to 0.025 approximately.

The aqueous phase (AP) comprises mainly water (i.e. at least 80% by volume of water) and optionally an alcohol, such as methanol, ethanol, isopropanol or butanol, and preferably ethanol.

Step 2) is preferably carried out by gradually introducing the fatty phase resulting from step 1) into the aqueous phase (AP).

During step 3), the O/W emulsion is maintained at a temperature greater than the temperature M_{P}.

Advantageously, the amount of colloidal solid particles present in the continuous aqueous phase is adjusted as a function of the volume average size of the droplets of fatty phase in the liquid state desired in the O/W emulsion, as measured by quasi-elastic dynamic laser light scattering.

In particular, the amount of colloidal solid particles within the O/W emulsion ranges from 35 mg to 75 mg of colloidal solid particles/g of fatty phase, and even more preferentially from 60 mg to 68 mg of colloidal solid particles/g of fatty phase.

The average diameter of the droplets of fatty phase in the liquid state preferably ranges from 350 nm to 950 nm approximately, and even more preferentially from 740 nm to 825 nm approximately.

The size distribution of the droplets of the fatty phase in the O/W emulsion is generally narrow (polydispersity <20% approximately).

The mechanical stirring of step 3) can be carried out using a dispersion device such as, for example, a dispersion device sold under the trade name Ultra-Turrax® T25 by the company Janke & Kunkel™ or Rayneri® and/or using a high-pressure microfluidizer, such as, for example, a microfluidizer sold under the trade name MS110 by Microfluidics™.

According to one particularly preferred embodiment of the invention, step 3) comprises a substep of stirring with a conventional dispersion device, then a substep of stirring with a microfluidizer.

The use of a microfluidizer makes it possible in particular to decrease the size of the droplets of fatty phase in the liquid state.

Step 4) is a resting step (without stirring) in order to induce a phenomenon of limited coalescence and to obtain a Pickering emulsion comprising monodisperse globules of fatty phase in the solid state, dispersed in the continuous aqueous phase.

The preceding step 3) made it possible to fragment the droplets of fatty phase in the liquid state into an average size much lower than D. The amount of oil/water interface is then higher than the surface capable of being covered by the colloidal solid particles. There is thus initially a “virgin” surface fraction, that is to say not protected by the colloidal solid particles. Thus, after the stirring has stopped (step 4)), the drops will coalesce and the total amount of interface will decrease. Since the adsorption of the colloidal solid particles is irreversible, the coalescence will stop when the droplets have reached a diameter equal to D (or greater than D if the particles do not form a monolayer or are not all adsorbed) and will be entirely covered. This phenomenon is a “partial coalescence” or “limited coalescence” phenomenon. The Pickering emulsion is stable over the course of several weeks and characterized by a narrow size distribution of the globules of fatty phase in the solid state. The colloidal solid particles may also serve as nucleation sites for initiating the subsequent mineralization step 5) (sol-gel process) and forming the silica shell.

During step 5), the addition of at least one precursor of silicon oxide and acid pH brings about the condensation of said precursor at the interface of the globules of fatty phase in the solid state and the formation of the shell.

The precursors of silicon oxide can be chosen from silicon alkoxides, and in particular from tetramethoxyorthosilane (TMOS), tetraethoxyorthosilane (TEOS), di methyldiethoxysilane (DM DES), (3-mercaptopropyl)trimethoxysilane, (3-aminopropyl)triethoxysilane, N-(3-trimethoxysilylpropyl)pyrrole, 3-(2,4-dinitrophenylamino)propyltriethoxy-silane, N-(2-aminoethyl)-3-aminopropyltrimethoxysilane, phenyltriethoxy-silane, methyltriethoxysilane, and a mixture thereof.

Among these precursors, TEOS is particularly preferred. These precursors can be totally or partially substituted with silicate sols.

The thickness of the shell depends on the amount of precursors of silicon oxide used during step 5) and on the diameter of the dispersed globules of the fatty phase in the solid state. The concentration of precursors of silicon oxide is expressed in mol/l (i.e. in M), relative to the total surface area in m² of the dispersed globules of the fatty phase in the solid state of the O/W emulsion.

According to one preferred embodiment of the invention, the amount of silicon oxide precursor(s) ranges from 0.001 to 1 M/m² approximately and even more preferentially from 0.005 to 0.1 M/m² approximately of surface area of the globules of the fatty phase in the solid state.

In order to achieve the largest thicknesses of the shell, step 5) can be carried out several times until the desired thickness is obtained.

When the shell of the material in accordance with the invention comprises, in addition to the silicon oxide, a metal oxide, at least one precursor of a metal oxide of formula MeO₂ is then also added to the continuous aqueous phase of the O/W emulsion, said precursor being chosen from the alkoxides, chlorides and nitrates of the metals Me chosen from Zr, Ti, Th, Nb, Ta, V, W and Al.

When they are used, the amount of these precursors of metal oxide of formula MeO₂ ranges from 0.001 to 1 M/m², and preferentially from 0.01 to 0.6 M/m² of surface area of the globules of the fatty phase in the solid state.

The pH of the aqueous phase during step 5) preferably ranges from 0.01 to 4, and even more preferentially from 0.05 to 2.1.

The acid used to adjust the pH of the aqueous phase can be chosen from mineral acids and organic acids, among which mention may in particular be made of hydrochloric acid, acetic acid, nitric acid, perchloric acid or sulfuric acid.

Hydrochloric acid is preferred.

In addition to the acid and the precursor of silicon oxide, a surfactant SA₁ is also added during step 5).

The SA₁ surfactant is preferably chosen from cationic surfactants, such as hexadecyltrimethylammonium bromide (CTAB).

It makes it possible to catalyse the condensation reaction and to control the thickness of the shell of the submicrometric capsules constituting the material.

The SA₁ surfactant is preferably used in a proportion of from 0.001 g to 0.1 g approximately, per gram of precursor of silicon oxide, and even more preferentially from 0.004 g to 0.05 g approximately, per gram of precursor of silicon oxide.

Step 5) is preferably carried out by first adding the SA₁ surfactant and then the precursor of silicon oxide to the continuous aqueous phase of the emulsion.

The precursor of silicon oxide is preferably added dropwise to the continuous aqueous phase of the emulsion.

During step 6), the material in accordance with the invention can be separated from the aqueous phase and recovered by any conventional separation technique known to those skilled in the art, such as filtration, centrifugation and/or the use of a sieve. It is then preferably washed, for example with water, then dried, for example by freeze-drying, to give a powder.

The material obtained at the outcome of step 5) or 6) is stable with respect to storage for several months, provided that the storage temperature is lower than the melting point M_{P} of the fatty phase enclosed in the shell.

The process of the invention may also comprise a step 4′), after step 4), during which an additional amount of colloidal solid particles (optionally functionalized so as to make them hydrophobic as described above) is added. The additional amount serves to increase the degree of coverage of the droplets so as to improve the stability thereof. The additional amount of colloidal solid particles that is added preferably ranges from 0.006 to 0.012 g approximately of colloidal solid particles/g of fatty phase. This additional amount of optionally functionalized colloidal solid particles may make it possible to prevent the aggregation of the globules of fatty phase in the solid state and may enable the storage of the emulsion. This may also make it possible to improve the heat resistance of the emulsion.

The process may also comprise a step i), prior to step 1), for preparing superparamagnetic nanoparticles functionalized with at least one fatty acid as defined in the invention. Step i) may in particular comprise a substep i-1) of preparing the superparamagnetic nanoparticles, and a substep i-2) of functionalizing said nanoparticles with at least one fatty acid as defined in the invention.

Substep i-1) of preparing the superparamagnetic nanoparticles may comprise a size sorting or selection process based on successive phase separations. Thus, at the outcome of substep i-1), the superparamagnetic nanoparticles may be homogeneous with respect to size.

In particular, when the superparamagnetic nanoparticles are maghemite nanoparticles, substep i-1) can be carried out in accordance with the process described by Massart et al. [IEEE Transactions on Magnetics, 1981, 17, 2, 1247-1248]. Substep i-1) comprises in particular the preparation of magnetite particles, the oxidation thereof to maghemite (e.g. in the presence of ferric nitrate (FeNO₃)) and a size selection (or sorting) process. The size selection (or sorting) process can in particular be carried out according to the procedure described by Massart et al. [Journal of Magnetism and Magnetic Materials, 1995, 149, 1-2, 6-7]. The maghemite nanoparticles are then homogeneous with respect to size (i.e. reduced size distribution).

The material in accordance with the invention may be used in the form of a powder or of a dispersion in the solvent in order to deliver the substance(s) of interest present in the solid fatty phase enclosed in the silicon oxide-based shell.

A subject of the invention is thus also the use of a material in accordance with the invention and as described above, for the magnetically stimulated delivery of at least one substance of interest.

The material in accordance with the invention is consequently intended to be used for the magnetically stimulated delivery of at least one substance of interest.

The delivery of the substance of interest is obtained by breaking of the shell under the effect of a radio frequency alternating magnetic field, leading to a local increase (i.e. in the core of the capsule) of the temperature to a delivery temperature T_{D} such that T_{D}>M_{P}.

The frequency of the radio frequency alternating magnetic field may be between 3 kHz and 30 MHz approximately (low-frequency field), between 30 and 300 MHz approximately (high-frequency or VHF field) or between 300 MHz and 3 GHz approximately (ultra-high frequency or UHF field). For in vivo applications, the frequency of the field is preferably between 30 and 900 KHz approximately.

The intensity of the radio frequency alternating magnetic field can range from 2 to 40 kA/m approximately (i.e. a magnetic induction B of between 2.5 and 50 mT).

According to one advantageous embodiment, in particular for in vivo applications, the product of the frequency multiplied by the intensity of the radio frequency alternating field is at most 5×10⁹ A/m/s.

The radio frequency alternating magnetic field can be applied by any appropriate means known to those skilled in the art, in particular using a magnetic resonance imaging (MRI) apparatus.

By way of example, and when the substance of interest is a drug, the crystallizable oil present in the fatty phase is preferably chosen from crystallizable oils having a melting point of greater than approximately 36° C. Thus, when said material is incorporated into a pharmaceutical composition and this composition is administered to a patient, for example orally, the ingested composition will be at body temperature, in general 37° C. In order to bring about the breaking of the capsule, the whole body of the patient, or else just one part, may be placed in a magnetic field of suitably chosen frequency and amplitude, leading to the microscopic/local heating of the fatty phase and the volumetric expansion thereof, and thus the breaking of the silica shell and the delivery of the drug.

According to another example, the substance of interest is a cosmetic active ingredient and the material is part of the components of a cosmetic composition for topical application, such as a powder, a cream or a gel. The application of a magnetic field of suitably chosen frequency and amplitude can gradually induce the local heating of the fatty phase of the material at a temperature greater than M_{P} (without burning the skin) and allow slow and controlled incorporation of the substance of interest into the pores of the skin, in particular while avoiding any bursting of the capsules.

If the cosmetic composition is in the form of a powder, the application of a magnetic field of suitably chosen frequency and amplitude may be accompanied by a change in texture (conversion of the powder into a composition having a fatty feel due to the breaking of the shell) while avoiding any projection of powder into the eyes or the sensitive areas of the human body.

By way of examples of use of the material in accordance with the invention, mention may in particular be made of the use of said material in the medical imaging field, for example as contrast agent for magnetic resonance imaging (MRI). Indeed, the superparamagnetic nanoparticles encapsulated can provide MRI image contrast properties by modifying the relaxation time of the hydrogen nuclei of the water and of the fats in the organs. Once in the organism, the material may in addition be guided to an organ or in particular a tumour by virtue of the application of a static and non-homogeneous magnetic field (field gradient), which is another advantage of the remote magnetic control of the capsules (in addition to the magnetically induced release of active agent). Said material can thus be used for magnetic guidance to a target organ or a target tumour.

A subject of the invention is also the use of the material as described above, as an ingredient, for the preparation of pharmaceutical, cosmetic or food products, and also the pharmaceutical, cosmetic or food products containing, as ingredient, at least one material in accordance with the invention.

These compositions may contain the conventional pharmaceutical, cosmetic or food supports well known to those skilled in the art, and also one or more surfactants intended to promote the release of the liquid fatty phase during the breaking of the capsule.

The present invention is illustrated by the following exemplary embodiments, to which it is not however limited.

### EXAMPLES

The starting materials used in the examples which follow are listed below:
cetyltrimethylammonium bromide (CTAB), purity 98%, the company Sigma-Aldrich;
diethyl ether, the company Sigma-Aldrich;
solution of hydrochloric acid at 37% by weight, the company Sigma-Aldrich;
technical-grade methanol, the company Sigma-Aldrich;
solution of nitric acid at 69% by weight, the company Sigma-Aldrich;
technical-grade acetone, the company Sigma-Aldrich;
solution of ammonium hydroxide at 30% by weight, the company Sigma-Aldrich;
non-hydrated ferric nitrate, purity 98%, the company Alfa Aesar;
solution of ferric chloride at 45% by weight, the company Sigma-Aldrich;
ferrous chloride tetrahydrate, purity 98%, the company Alfa Aesar;
oleic acid, purity 90%, the company Sigma-Aldrich;
stearic acid, purity 95%, the company Sigma-Aldrich;
chloroform, the company Sigma-Aldrich;
silica nanoparticles 7 nm in diameter, sold under the name Aerosil™ A380 by the company Evonik Degussa;
n-eicosane (C₂₀H₄₂), purity 99%, melting point=36° C., the company Aldrich; and
tetraethoxyorthosilane (TEOS), the company Sigma-Aldrich.

These starting materials were used as received from the producers, without additional purification.

The materials obtained were characterized by several techniques described below.

In order to determine the fatty acid concentration of the functionalized iron oxide nanoparticles, thermogravimetric analyses (TGAs) were carried out using a thermal balance sold under the trade name Setaram Instrumentation™ using a flow of air and by heating the sample from 20 to 800° C. with a temperature increase of 10° C. per minute.

The materials were observed using a scanning electron microscope (SEM) sold under the reference TM-1000 by the company Hitachi. The material analysed was, beforehand, either dried at ambient temperature, or freeze-dried for 12 h at −80° C. using a freeze-dryer sold under the name Alpha 2-4 LD Plus by the company Christ. All the materials were covered with gold before being observed by SEM.

The magnetic hyperthermia experiments were carried out using an induction soldering apparatus sold under the trade name Minimax Junior™ 1 TS from the Italian company Seit Elettronica resold by the company Maxmatic. The apparatus used comprises a MOSFET (Metal Oxide Semiconductor Field Effect Transistor) 3.5 kW generator producing a quasi-sinusoidal alternating magnetic field at a radio frequency of 755 kHz in a resonating circuit comprising a 4-turn induction coil (internal diameter of 50 mm and height of 32 mm) refrigerated by internal circulation of cold water (i.e. inside the conducting wires). The intensity of the alternating magnetic field was estimated at 10.2 kA/m at total power (747 V, 234 amps) by means of FEMM (Finite Element Model Magnetics) software for simulating magnetism problems leaving finite elements (http://www.femm.info/).

### Example 1: Production and Characterizations of Materials in Accordance with the Invention

1) Production of Materials in Accordance with the Invention

1.1) Preparation of the Monodisperse Superparamagnetic Nanoparticles Functionalized with a Fatty Acid

Superparamagnetic nanoparticles of maghemite of formula γ-Fe₂O₃ were prepared according to the process described by Massart et al. [IEEE Transactions on Magnetics, 1981, 17, 2, 1247-1248].

Firstly, polydisperse nanocrystals of magnetite of formula Fe₃O₄ (or FeO.Fe₂O₃) were prepared in the aqueous phase by alkaline coprecipitation. To do this, 180 g of a ferrous chloride and 367 ml of a 45% ferric chloride solution (i.e. 4.1 M) were introduced, according to the non-stoichiometric molar proportions 0.9:1.5, into a solution comprising 100 ml of concentrated HCl at 37% (approximately 12.2 M) diluted in 500 ml of water. The resulting solution was then diluted with water so as to form a total volume of aqueous solution of 3 litres. The resulting aqueous solution was placed under vigorous mechanical stirring (approximately 800 revolutions per minute) and 1 litre of a concentrated aqueous ammonia solution at 30% was added as rapidly as possible in order to enable the coprecipitation. A black precipitate characteristic of magnetite was thus obtained. The resulting suspension was stirred for 30 minutes and decanted using a permanent ferrite magnet sold under the trade name Calamit Magneti™ having the dimensions 152×101×25.4 mm³, until the supernatant was colourless (at least 10 minutes). The magnet was used to accelerate the extraction of the supernatant then suction thereof by means of a vacuum flask. After washing the precipitate with 1 litre of distilled water, then again the magnetic sedimentation, the flocculate was acidified with a solution comprising 360 ml of nitric acid at 69% (15 M) diluted in 1.6 litres of distilled water. After 30 minutes of stirring, the suspension, that was now acidic, was again decanted on the permanent magnet until a clear supernatant was obtained, which supernatant was subsequently suctioned and then eliminated.

Secondly, all of the polydisperse nanocrystals of colloidal magnetite Fe₃O₄ previously obtained were oxidized to maghemite of formula γ-Fe₂O₃ by addition of a solution comprising 323 g of ferric nitrate (FeNO₃) diluted in 800 ml of water brought to boiling (90-100° C.) with mechanical stirring. The resulting suspension turned brick red, the characteristic colour of maghemite. The precipitate was decanted on the permanent magnet, the supernatant was suctioned, then 360 ml of nitric acid at 69% diluted in 1.6 litres of distilled water were added thereto. In order to remove all the excess ferric nitrate ions, the suspension was washed once with 1 litre of acetone (stirring for 10 minutes, magnetic decanting then suctioning of the supernatant), then twice with 500 ml of diethyl ether (stirring for 10 minutes, magnetic decanting, then suctioning of the supernatant). Finally, the organic solvents were evaporated off by mechanical stirring under a suction hood, the maghemite particles having been redispersed beforehand in water acidified to a pH of approximately 2 by addition of nitric acid, so as to form a stable but polydisperse suspension of maghemite nanoparticles, with sizes ranging from 5 nm to 20 nm approximately.

Thirdly, the maghemite nanoparticles obtained were subjected to a size selection (or sorting) process as described by Massart et al. [Journal of Magnetism and Magnetic Materials, 1995, 149, 1-2, 6-7]. This process made it possible to reduce the polydispersity of maghemite nanoparticles. This particle-size sorting process is well known to those skilled in the art. It is based on phase separation by fractionation. To do this, an excess nitric acid solution (NHO₃ at 15 M) was added to the stock suspension of polydisperse maghemite nanoparticles, as prepared above. This thus made it possible to decrease the pH (initially at 2) down to 0.8 and to induce an increase in the ionic strength and thus the formation of an upper phase, the supernatant, more dilute in terms of solid fraction and containing nanoparticles of smaller sizes, and of a more concentrated lower phase, attracted by the magnet, containing particles of larger sizes. The two phases were then separated after decanting by means of a magnet as described above and suctioning of the upper phase. By repeating these steps on each of the fractions, it was possible to obtain a fraction comprising sorted maghemite nanoparticles having a size of between 12 and 15 nm approximately.

The nanoparticles as prepared above were then functionalized either with stearic acid, or with oleic acid.

With regard to the nanoparticles functionalized with oleic acid, a mixture comprising the following molar proportions of oleic acid/aqueous ammonia/iron of 1/1/5 was heated at approximately 60° C. for 30 minutes with mechanical stirring. The resulting mixture separated into a foaming aqueous phase and a brown-black-coloured hydrophobic paste, which was sedimented on the permanent magnet after cooling to ambient temperature (approximately 20° C.) then washed three times with methanol and dried under vacuum for 30 min in order to remove as much water as possible.

With regard to the nanoparticles functionalized with stearic acid, a mixture comprising the molar proportions of stearic acid/aqueous ammonia/iron of 1/1/5 was heated at approximately 70° C. for 30 minutes with mechanical stirring. The resulting mixture separated into a foaming aqueous phase and a brown-black-coloured hydrophobic paste, which was sedimented on the permanent magnet after cooling to ambient temperature (approximately 20° C.), then washed three times with methanol and dried under vacuum for 30 min in order to remove as much water as possible.

The functionalization conferred a lipophilic nature on the maghemite nanoparticles, thus making it possible to incorporate them into a fatty phase as defined in the invention. Thus, the functionalized, size-sorted maghemite nanoparticles will form a stable suspension in the crystallizable oil when it is in the liquid form, and remain at the core of the submicrometric capsules during their production.

The Specific Absorption Rate or SAR (in watts per gram) of the functionalized maghemite superparamagnetic nanoparticles was measured at approximately 280 W/g in water under the alternating magnetic field conditions used (10.2 kA/m at 755 KHz). This absorption rate decreases in eicosane to approximately 8 W/g (maghemite nanoparticles functionalized with oleic acid) or 6 W/g (maghemite nanoparticles functionalized with stearic acid) probably due to the immobilization of the magnetic nanoparticles in the wax, which greatly reduces the thermal power dissipated by the oscillation of the magnetic moments (phenomenon amplified with stearic acid which is also crystalline at ambient temperature).

Thermogravimetric analyses (TGAs) showed that the maghemite nanoparticles functionalized with oleic acid comprise approximately 130 mg of oleic acid per gram of solid paste of maghemite nanoparticles functionalized with oleic acid and the nanoparticles functionalized with stearic acid comprise approximately 220 mg of stearic acid per gram of solid paste of maghemite particles functionalized with stearic acid.

1.2) Preparation of functionalized colloidal solid particles

1.18 g of Aerosil™ A380 silica nanoparticles were dispersed in 100 ml of distilled water, using an ultrasonic bath. 22.4 mg of CTAB were subsequently added to this dispersion, this amount representing approximately a factor of 0.65 of the critical micelle concentration of CTAB (CMC=0.9×10⁻³ mol/l). Since the surface of the silica nanoparticles is negatively charged, the CTAB (cationic surfactant) adsorbs at the surface of the silica nanoparticles and thus makes it possible to confer a hydrophobic nature thereon. This hydrophobic nature allows them to stabilize the fatty phase-continuous aqueous phase interface of the emulsion during its preparation. A dispersion of surface-functionalized silica nanoparticles in an aqueous phase was obtained. The weight of surfactant/weight of colloidal solid particles ratio by weight was approximately 0.019.

1.3) Preparation of the Emulsions

In order to prepare two emulsions, the compositions of which are specified in table 1 below, a given amount of solid paste of maghemite nanoparticles functionalized with oleic acid or with stearic acid, as prepared in example 1.1), was added to 18 g of eicosane (crystallizable oil), in order to obtain a suspension comprising a final concentration of iron oxide of approximately 12 g/l. The resulting fatty phase was heated to approximately 55° C. in order to melt the eicosane [step 1)] in which the functionalized, size-sorted supermagnetic nanoparticles form a homogeneous and clear suspension.

Analyses by dynamic light scattering made it possible to show that the maghemite nanoparticles functionalized with oleic acid in suspension in the fatty phase have an average hydrodynamic size of approximately 25 nm with a polydispersity index (PDI) of approximately 0.36 (measurement carried out by QELS on a suspension at 4 g/l) and the maghemite nanoparticles functionalized with stearic acid in suspension in the fatty phase have an average hydrodynamic size of approximately 24 nm with a polydispersity index (PDI) of approximately 0.4 (measurement carried out by QELS on a suspension at 2 g/l). This shows that the maghemite nanoparticles are individually dispersed (i.e. no formation of aggregates or clusters) and coated with a self-assembled monolayer of fatty acid molecules ensuring effective stearic repulsion against the Van der Waals and magnetic dipolar forces between the grains.

In parallel, the aqueous phase comprising silica nanoparticles functionalized with CTAB, in suspension as prepared in example 1.2), was heated to approximately 55° C. A given amount of fatty phase as prepared above was then gradually incorporated into a given amount of abovementioned aqueous phase [step 2)] and the whole mixture was vigorously stirred and homogenized using a stirrer sold under the name Ultra-Turrax™ T25 by the company Janke & Kunkel™, equipped with an S25 N-25F dispersion tool, at a speed of approximately 20 000 revolutions for 1 min. In order to obtain smaller droplets of fatty phase, the resulting mixture was transferred into a high-pressure microfluidizer sold under the trade name MS110 by Microfluidics™ and microfluidized for approximately 30 seconds at a pressure of approximately 95 MPa. During the preparation of the emulsion, the latter was maintained at 55° C., in order to avoid any crystallization of the crystallizable oil [step 3)].

TABLE 1

| | Amount of | Amount of | Amount of | Amount of silica |
|---|---|---|---|---|
| | functionalized | fatty | aqueous | nanoparticles per |
| O/W | maghemite | phase in the | phase in the | gram of fatty phase |
| emul- | nanoparticles | emulsion | emulsion | in the emulsion |
| sion | (in g) | (in g) | (in g) | (in mg) |
| E-OA | 0.31 | 18.31 | 101.2024 | 64 |
| E-SA | 0.33 | 18.33 | 101.2024 | 64 |

The average diameter of the droplets of fatty phase in the liquid state was approximately 740 nm for the E-OA emulsion and approximately 900 nm for the E-SA emulsion.

The resulting emulsion was then left to stand in an oven at 55° C. for 10 min in order to reveal the limited coalescence phenomenon. Once cooled to a temperature below the melting point of the crystallizable oil (eicosane) [step 4)], a small amount of silica nanoparticles functionalized with CTAB (solution of 0.17 g of nanoparticles functionalized with CTAB, dispersed in 4.8 ml of water) was added to the E-OA emulsion [step 4′)]. The addition of this supplementary amount of colloidal solid particles makes it possible to prevent the aggregation of the wax particles and allow the storage of the emulsion at ambient temperature. Approximately 119.5 g of each of the emulsions E-OA and E-SA comprising globules of fatty phase in the solid state, dispersed in a continuous aqueous phase, were thus obtained.

1.4) Preparation of the Submicrometric Capsules in Accordance with the Invention: Formation of the Acidic Shell (Mineralization Step)

In this step [step 5)], the formation of the silica shell around the globules of fatty phase in the solid state was carried out.

The two emulsions E-OA and E-SA were diluted from 18% by weight to 2% by weight and the pH of the emulsions was adjusted to approximately 0.2, that is to say to a value below the isoelectric point of silica, by addition both of 7 g of a solution of hydrochloric acid at 37% by weight (approximately 12.2 M) and of 80 g of an aqueous solution containing 0.21 g of CTAB.

5 g of TEOS were then added dropwise to the two emulsions in order to reach the amount denoted in Table 2 below. During the addition, the solution was placed under magnetic stirring at a speed of 450 rpm, this speed not modifying the size distribution of the drops. The resulting dispersion was placed in 50 ml test tubes overnight with continuous stirring on a wheel at 25 rpm in a thermostated chamber at 20° C. so as to allow the silica shell to form (mineralization).

At the end of the mineralization, submicrometric capsules of silica were recovered after several cycles of centrifugation-redispersion several times in distilled water [step 6)]. The material obtained was stored in pure water for several months. No modification of the submicrometric capsules was observed during this period.

TABLE 2

| | Amount of CTAB | Amount of |
|---|---|---|
| Emulsion | per g of TEOS (in g) | TEOS (in M/m²) |
| E-OA | 0.042 | 0.039 |
| E-SA | 0.042 | 0.043 |

2) Results of the Characterizations

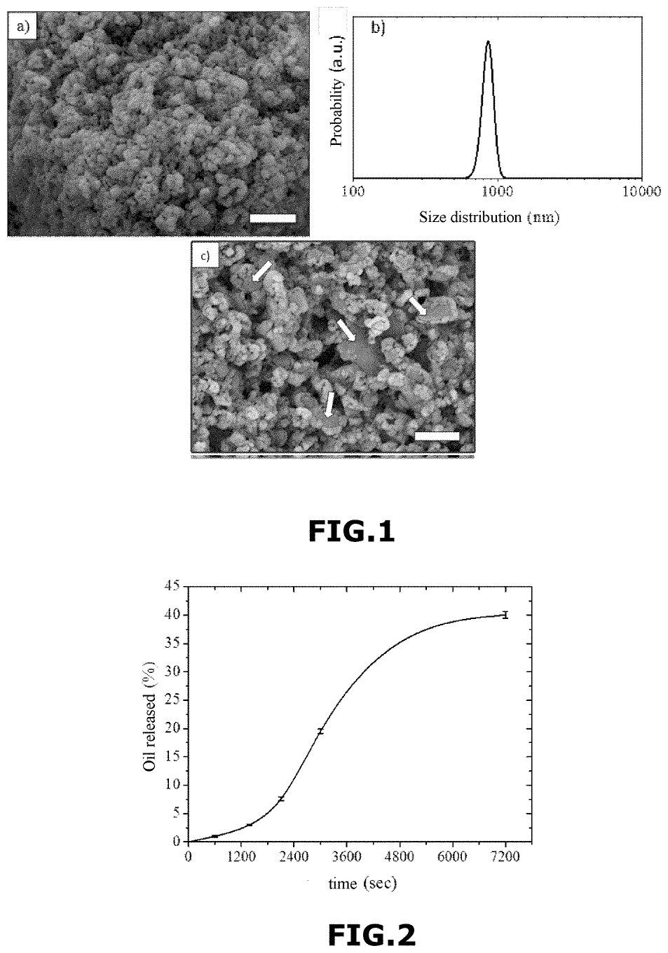

The appended FIG. 1 represents an SEM photograph taken during the observation of a material in accordance with the invention obtained by mineralization of the E-SA emulsion (FIG. 1a), then its size distribution showing a material comprising particles having a submicrometric size centred around 825 nm (FIG. 1b), and finally an SEM photograph taken during the observation of a material in accordance with the invention obtained by mineralization of the E-SA emulsion after breaking of the envelope by application of a radio frequency of alternating magnetic field (FIG. 1c). In FIGS. 1a and 1c, the scale bar represents 5 μm and on FIG. 1c, the white arrow points to the breaking zone brought about by the expansion of the fatty phase.

### Example 2: Release Profile of the Materials in Accordance with the Invention

In this example, the breaking of a material in accordance with the invention obtained by mineralization of the E-SA emulsion is illustrated.

The material was exposed to an alternating magnetic field at a radio frequency of approximately 755 kHz and an intensity of approximately 10.2 kA/m for a variable time: 600, 1380, 2100, 3000 and 7200 seconds.

FIG. 2 shows the amount of crystallizable oil released (as percentage) as a function of the time of application of the alternating magnetic field (in seconds).

According to FIG. 2, it appears that approximately 20% of the oil is released after 50 minutes (3000 s) of application of a radio frequency alternating magnetic field of 10.2 kA/m at 755 kHz, up to reaching 40% after 2 hours (7200 s) of application of a radio frequency alternating magnetic field of 10.2 kA/m at 755 kHz.

It is possible to accelerate the rate of release of the oil and thus that of a substance of interest by increasing the amount of superparamagnetic nanoparticles in the core of the capsules (modification of the fatty acid for example) and/or their specific absorption rate by means of a modification of the shape and of the type of superparamagnetic nanoparticles used, for example with “nanocubes” or “nanoflowers”, that is to say multi-core nanoparticles, which can achieve SAR values of more than 1000 W/g.

1. Material in the form of solid particles containing a fatty phase that is solid at the storage temperature of said material and a continuous shell comprising:
at least one silicon oxide and enclosing said fatty phase, said fatty phase comprising a crystallizable oil having a melting point (M_{P}) of less than 100° C. and at least one substance of interest, wherein said material is submicrometric and in that the fatty phase also comprises superparamagnetic nanoparticles surface-functionalized with at least one fatty acid.

2. Material according to claim 1, wherein the crystallizable oil is chosen from paraffins, triglycerides, fatty acids, rosins, waxes, hydrogenated plant oils and also mixtures thereof, and synthetic bitumens.

3. Material according to claim 1, wherein the diameter of the particles ranges from 400 nm to 900 nm.

4. Material according to claim 1, wherein the particles constituting said material are monodisperse.

5. Material according to claim 1, wherein the diameter of the superparamagnetic nanoparticles contained in the fatty phase ranges from 10 to 20 nm.

6. Material according to claim 1, wherein the superparamagnetic nanoparticles are maghemite nanoparticles.

7. Material according to claim 1, wherein the fatty acid is chosen from arachidic acid, stearic acid, oleic acid, palmitic acid, myristic acid, lauric acid, capric acid and caprylic acid.

8. Material according to claim 1, wherein the functionalized superparamagnetic nanoparticles represent from 0.2% to 3% by weight of the total weight of the fatty phase.

9. Material according to claim 1, wherein the thickness of the silica shell ranges from 30 to 50 nm.

10. Process for producing a material as defined in claim 1, wherein said process comprises at least the following steps:
1) preparing a fatty phase in the liquid state comprising a crystallizable oil in the liquid state having a melting point M_{P} of less than approximately 100° C., at least one substance of interest and superparamagnetic nanoparticles functionalized with at least one fatty acid;
2) bringing said fatty phase in the liquid state of step 1) into contact with an aqueous phase (AP) brought beforehand to a temperature T_{AP} such that T_{AP} is greater than M_{P}, said aqueous phase (AP) containing colloidal solid particles;
3) subjecting the liquid mixture resulting from step 2) to mechanical stirring so as to obtain an oil-in-water (O/W) emulsion formed of droplets of fatty phase in the liquid state dispersed in a continuous aqueous phase and in which the colloidal solid particles are present at the interface formed between the continuous aqueous phase and the dispersed droplets of fatty phase;
4) leaving said O/W emulsion to stand and then cooling it to a temperature T_{O/W} such that T_{O/W} is less than M_{P} so as to bring about the solidification of the fatty phase and to obtain an O/W emulsion formed of globules of fatty phase in the solid state, said globules being dispersed in the continuous aqueous phase;
5) forming a shell comprising at least one silicon oxide around each of said globules by addition, to the continuous aqueous phase of the O/W emulsion of step 4), and with mechanical stirring, of at least one precursor of silicon oxide, of a surfactant SA₁ and of a sufficient amount of at least one acid to bring the aqueous phase to a pH of less than or equal to 4 so as to obtain said material;
6) optionally, separating said material from the aqueous phase.

11. Process according to claim 10, wherein the colloidal solid particles are chosen from nanoparticles of silicon oxide.

12. Process according to claim 10, wherein the colloidal solid particles are surface-functionalized so as to make them more hydrophobic by adsorption of molecules of a surfactant SA₂ at their surface by electrostatic bonds.

13. Process according to claim 10, wherein the magnetic stirring of step 3) is carried out using a dispersion apparatus and/or using a high-pressure microfluidizer.

14. At least one substance of interest for magnetically stimulated delivery, wherein said at least one substance of interest is within the material as defined in claim 1.

15. A contrast agent or for magnetic guidance to a target organ or target tumour in the medical imaging field, wherein said contrast agent is within the material as defined in claim 1.
